(19) United States
(12) Patent Application Publication
(10) Pub. No.: US 2010/0114014 A1
(43) Pub. Date: May 6, 2010

(54) PHARMACEUTICAL COMPOSITIONS STABILISED IN GLASSY PARTICLES

(75) Inventor: Bruce Roser, Cambridge (GB)

Correspondence Address: KOLISCH HARTWELL, P.C., 200 PACIFIC BUILDING, 520 SW YAMHILL STREET, PORTLAND, OR, 97204, US

(73) Assignee: CAMBRIDGE BIOSTABILITY LIMITED, Cambridge (GB)

(21) Appl. No.: 12/089,386

(22) PCT Filed: Oct. 4, 2006
(86) PCT No.: PCT/GB2006/050312

§ 371 Date: Apr. 4, 2008

(30) Foreign Application Priority Data
Oct. 4, 2005 (GB) 0520129.8

(51) Int. Cl. A61M 5/315; A61K 9/14; A61P 43/00; A61M 5/178

(52) U.S. Cl. 604/60; 424/489; 604/218

(57) ABSTRACT

Present proposals for liquid-medium carriers for glass particles comprising pharmaceutical compositions have had problems associated with aggregation of the glass particles. This has been previously solved by matching the density of the particles and a liquid in which they are suspended. However, though this greatly constrains the choice of liquid carrier that can be used. An alternative solution to this problem has been found. By adding just a small amount of liquid (2) to the particles (1), a flowable mixture is formed of a creamy or paste-like consistency. Providing too much liquid (2) is not present, the particles (1) do not separate out and so the choice of liquid carriers available for use in compositions of this type is greatly increased.

This invention relates to a pharmaceutical composition comprising an active material stabilized in water-soluble glassy particles which are mixed with an inactive anhydrous carrier liquid in which the particles are insoluble so that the composition can be administered by injection to an animal or human patient. After injection, the glass particles dissolve in body fluids, releasing the active ingredient.

There have been a number of proposals for liquids suitable to be used as the carrier in such compositions. Essential requirements are that it should be totally non-toxic and that the particles should not dissolve in it. In tests with oily liquids meeting these requirements it was initially found that the particles tended to fall to the bottom of the liquid. To avoid this problem it has been more recently proposed to select a liquid which is particularly dense and to add even denser additives to the particles so as to match their density with the liquid. This density matching technique has shown considerable promise but the need to use a high density liquid greatly constrains the choice of liquid; and those liquids which meet the high density requirement tend to be unsatisfactory for other reasons.

The invention arose in an endeavour to meet the above problem.

It has now been found that by adding just a small amount of liquid to the particles, a flowable mixture is formed of a creamy or paste-like consistency depending on the amount of liquid added. Providing too much liquid is not present in relation to the amount of solids, the particles do not separate out and so the choice of liquids available for use in compositions of this type is greatly increased.

According to the invention there is provided a pharmaceutical composition comprising an active material stabilized in glassy particles which are mixed with a liquid so that they can be delivered to a patient by injection through the skin characterised in that the proportion of particles to liquid is sufficiently high to prevent settlement of the particles at the top or bottom of the liquid.

Expressed another way, the invention provides a pharmaceutical composition comprising an active material stabilized in glassy particles which are mixed with a carrier so that they can be delivered to a patient by injection through the skin characterised in that the carrier is selected, and present in a quantity such that, the mixture has a viscosity or thickness preventing separation of the particles from the carrier.

Because only a small quantity of liquid is required, the volume of the composition needing to be injected is minute as compared with conventional techniques.

It will be understood that, by using the invention, a very wide variety of liquids can be used, in contrast with previous technical thinking which constrained the choice of liquids to those whose densities could be matched to the glassy particles. This means that other desired characteristics can be sought from amongst the vast range of liquids which can now be used.

Liquids which wet the particles are presumed to have the effect that each particle becomes coated with a layer of liquid, effectively forming a lubricant between the particles to assist flow. However, experiments have shown that “wettability” is not an essential characteristic. When a sample of hydrophilic sugar glass particles was mixed vigorously with squalane oil, which is hydrophobic, a thin paste-like mixture was formed, which was too thick to allow separation of the solid and liquid constituents but which nevertheless flowed sufficiently freely to pass though a conventional hypodermic needle. Another hydrophobic liquid, known to be suitable for injection, is sesame seed oil.

Another possible benefit of employing the invention is that, because the injected composition is relatively viscous or even semi-solid, in contrast with conventional non-viscous liquids, it is believed that it will disperse in the body slowly, releasing its active ingredients over a number of days.

Some of the abovementioned materials, eg polyethylene glycols, range from being low viscosity liquids to solid waxes. This raises the possibility of heating a normally waxy polyethylene glycol to make it liquid, dispersing the glassy particles in it, and then allowing it to solidify. If this is done inside a needle, a waxy plug is created on solidification. Because waxes contract on cooling, the plug can easily be pushed out of the needle into the patient's body.

Thus, according to a second aspect of the invention, there is provided a pharmaceutical composition comprising an active material stabilized in glassy particles which are mixed with a carrier so that they can be delivered to a patient by injection through the skin characterised in that the carrier is a waxy or semi-solid material.

According to a third aspect of the invention there is provided a device for injecting into a patient a pharmaceutical preparation comprising an active ingredient preserved in glassy particles, the device including a hypodermic needle; characterised in that the particles are mixed with a settable carrier material which has been allowed to solidify or semi-solidify to form a body in the needle; and means for pushing the solid or semisolid body out of the needle and into a patient.

Two ways in which the invention may be performed will now be described by way of example with reference to the accompanying drawings in which:

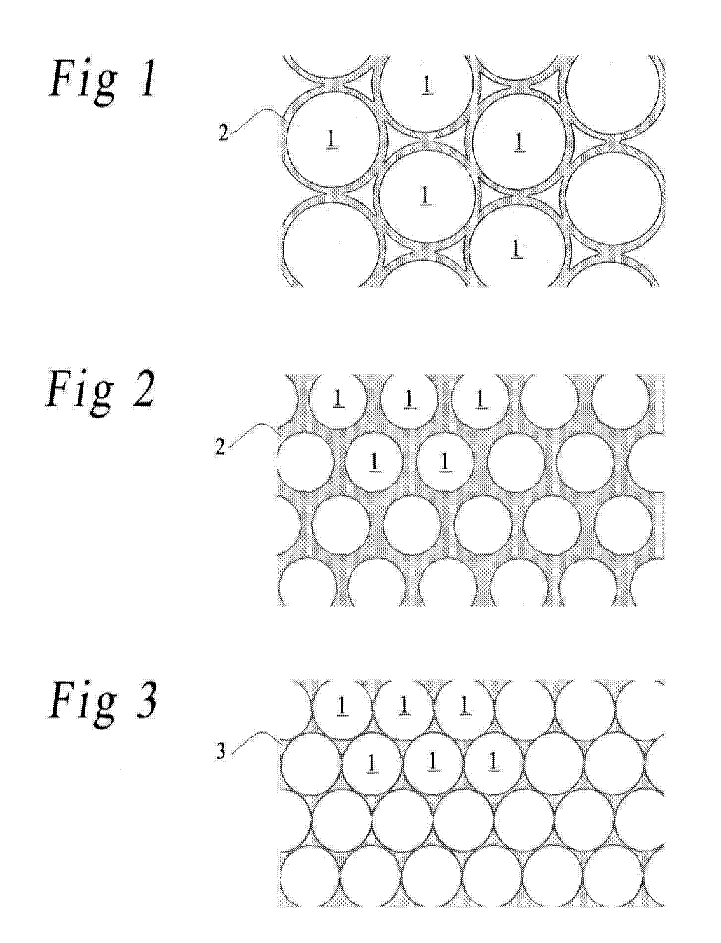

FIGS. 1 and 2 are magnified views of a first composition constructed in accordance with the invention and using a hydrophilic liquid;

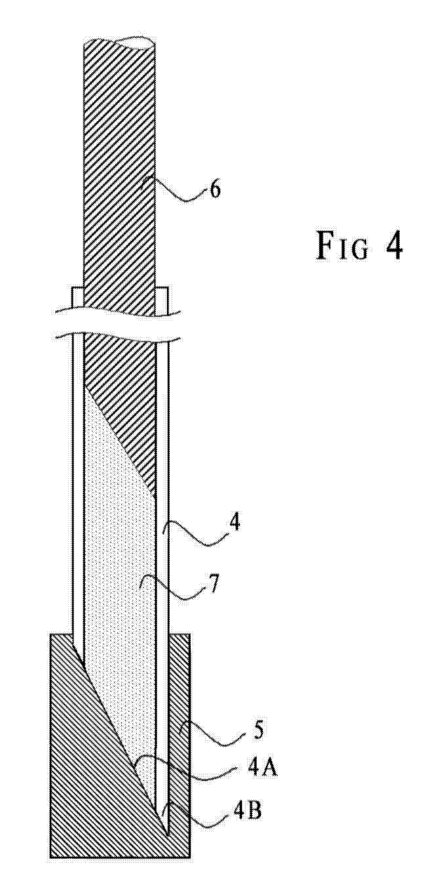

FIG. 3 is a similar view of a second composition using a hydrophobic liquid; and

FIG. 4 illustrates a delivery device constructed in accordance with the invention and pre-filled with a composition as shown in FIG. 1, 2 or 3.

Referring firstly to FIG. 1 there is shown, greatly magnified, a composition comprising spray dried particles 1 of a composition as described in patent specification WO 02/32402 and consisting of a mixture of sugar glass and an active ingredient such as a vaccine. To these particles is added a small quantity of a liquid polyethylene glycol. The intermolecular forces between the two materials (surface tension) cause the liquid to wet the particles, forming liquid layers on their surfaces. These liquid layers prevent the particles from touching and form a lubricant allowing the particles to slide relative to each other. Consequently, the mixture will flow freely eg through a hypodermic needle. It will be noted from FIG. 1 that spaces between the particles are not completely filled. This means that some air is trapped in the mixture, which could be a disadvantage.

FIG. 2 shows another composition, similar to that of FIG. 1, but with a higher proportion, by volume, of the liquid 2. In this composition the spaces between the particles are just filled, but with no excess liquid allowing solid and liquid phases to separate out. The benefits of the FIG. 2 composition are therefore obtained but without the problem of trapped air.

Another composition, illustrated in FIG. 3, was made by taking 1 ml of sugar-glass particles, adding 1 ml squalane, which is hydrophobic, and mixing vigorously using a glass rod for about five minutes. As mixing continued, the mixture became progressively more fluid, eventually attaining a thin creamy or paste-like consistency.

A feature of each of these compositions is that, if a substantially larger proportion of liquid were included, it would not be possible for the solid particles to remain suspended in it. They would eventually settle out and the mixture would need to be re-agitated before use.

Referring now to FIG. 4, there is shown an axial cross-section through a hypodermic needle 4, the lower end 4A of which is cut at a chamfered angle to form a sharp pointed end 4B. This is protected during storage by a plastic cap 5.

Inside the needle is a rod 6, the lower end 6A of which is chamfered at an angle equal to the angle of chamfer of the tube 4. The needle 4 is filled with a composition 7 as described above.

The components of FIG. 4 form part of a pre-filled injector which includes some mechanism, not shown, for pushing the rod 6 towards the pointed end of the needle so that the chamfered ends of the rod and needle coincide. In use, the cap 5 is removed, the needle is pushed into the patient and then the aforementioned mechanism operated to eject the composition 7.

1.-9. (canceled)

10. A pharmaceutical composition comprising an active material stabilized in glassy particles which are mixed with a liquid so that they can be delivered to a patient by injection through the skin characterised in that the proportion of particles to liquid is sufficiently high to prevent settlement of the particles at the top or bottom of the liquid.

11. The composition of claim 10 characterised in that the liquid is present in sufficient quantity to form a lubricant between adjoining particles.

12. The composition of claim 10 characterised in that substantially all of the liquid is held in association with the particles by the effects of inter molecular forces.

13. The composition of claim 10 characterised in that the liquid is present in sufficient quantity to form a lubricant between adjoining particles and in that substantially all of the liquid is held in association with the particles by the effects of inter molecular forces.

14. A composition according to any preceding claim characterised in that the liquid includes sesame seed oil or squalane or a polyethylene glycol.

15. A device for injecting a composition according to any one of claims 10 to 13 through the skin of a patient comprising a tube containing the composition, the tube having a pointed end which is sufficiently sharp and a diameter which is sufficiently small to allow penetration through the skin of a patient; and a plunger for expelling the composition from the pointed end of the tube after the latter has penetrated the skin of the patient.

16. A pre-filled syringe and needle containing a composition according to any one of claims 10 to 13.

17. A pharmaceutical composition comprising an active material stabilized in glassy particles which are mixed with a carrier so that they can be delivered to a patient by injection through the skin characterised in that the carrier is selected, and present in a quantity such that, the mixture has a viscosity or thickness preventing separation of the particles from the carrier.

18. A pharmaceutical composition comprising an active material stabilized in glassy particles which are mixed with a carrier so that they can be delivered to a patient by injection through the skin characterised in that the carrier is a semisolid.

19. A device for injecting into a patient a pharmaceutical preparation comprising an active ingredient preserved in glassy particles, the device including a hypodermic needle; characterised in that the particles are mixed with a settable carrier material which has been allowed to solidify or semi-solidify to form a body in the needle; and means for pushing the solid or semisolid body out of the needle and into a patient.
